(19) United States
(12) Patent Application Publication
(10) Pub. No.: US 2024/0384308 A1
(43) Pub. Date: Nov. 21, 2024

(54) METHODS FOR THE BIOTECHNOLOGICAL PRODUCTION OF ALDEHYDE MIXTURES

(71) Applicant: SYMRISE AG, Holzminden (DE)

(72) Inventors: Uwe BORNSCHEUER, Greifswald (DE); In Jung KIM, Greifswald (DE); Thomas BAYER, Graz (AT); Yannik BRACK, Greifswald (DE)

(73) Assignee: SYMRISE AG, Holzminden (DE)

(21) Appl. No.: 18/687,555

(22) PCT Filed: Aug. 23, 2022
(86) PCT No.: PCT/EP2022/073488

§ 371 Date: Feb. 28, 2024

(30) Foreign Application Priority Data
Aug. 31, 2021 (EP) 21193986.3

(51) Int. Cl. C12P 7/26; C12N 1/20; C12N 9/02; C12R 1/19

(52) U.S. Cl. CPC C12P 7/26; C12N 1/20; C12N 9/0008; C12N 9/0069; C12R 2001/19; C12Y 102/01003

(57) ABSTRACT

The present invention relates to biotechnological methods for producing saturated as well as unsaturated aldehydes, as well as mixtures thereof with at least one alpha-dioxygenase and at least one aldehyde dehydrogenase. The method can be carried out either fermentatively, as biotransformation or enzymatically. Furthermore, the present invention relates to a vector system, as well as sequences and recombinant microorganisms comprising/encoding enzymes which can be used to produce the aldehydes and mixtures according to the invention. Further, the present invention relates to compositions obtained by the methods according to the present invention

### TECHNICAL FIELD

The present invention relates to biotechnological methods for the production of saturated and unsaturated aldehydes, as well as mixtures thereof, with the aid of at least one alpha-dioxygenase and at least one aldehyde dehydrogenase. The method can be carried out either fermentatively, as a biotransformation or enzymatically. Furthermore, the present invention relates to a vector system, as well as sequences and recombinant microorganisms comprising/encoding enzymes which can be used to produce the aldehydes and mixtures according to the invention. Further, the present invention relates to compositions obtained by the methods according to the present invention.

### BACKGROUND TO THE INVENTION

In the field of industrial recovery of flavorings, there is a constant need for efficient and cost-effective ways of synthesizing flavorings. One example of such flavorings are (un) saturated aldehydes. An exemplary aldehyde is 8Z-tetradecenal or 1-decenal. This compound class is mainly found in meat preparations and citrus oils. However, no generally applicable biotechnological method for the production of these aldehydes and the corresponding acids has yet been described.

Processes for the production of unsaturated aldehydes are known from the prior art. For example, 8Z, 11Z-heptadecadienal can be obtained by chemical oxidation of the unsaturated alcohol (JP 63233914). Various aldehydes can also be produced in a known manner from fatty acids by alpha-oxidation using plant enzymes, as described in WO 2012/025629 A1. The use of algal biomass for the production of unsaturated aldehydes is also known (“Concise synthesis of (8Z, 11Z, 14Z)-8, 11, 14-heptadecatrienal, (7Z, 10Z,3Z)-7,10,13-hexadecatrienal, and (8Z, 11Z)-8, 11-heptadecadienal, components of the essential oil of marine green alga Ulva pertusa”, Biosci Biotechnol Biochem. 2005 Jul;69 (7): 1348-52). Processes for the production of 4,7,10, 13, 16-nonadecapentaenal or 3,6,9, 12,15, 18-heneicosahexaenal are also known (Preparation of halopolyenes and their intermediates, JP05000974A).

Furthermore, methods using aldehyde dehydrogenases in general are known to oxidize aldehydes to acids (Takeru Ishigeet al., Appl. Environ. Microbiol. 2000, 66, 3481-3486; Tomohisa Kato et al., Extremophiles 2010, 14, 33).

However, the above-mentioned chemical methods and methods based solely on the use of a very specific enzyme have the disadvantage that they do not provide access to a comprehensive spectrum of aldehydes or mixtures thereof. Furthermore, some of the methods are based on a reaction with chemical reagents and catalysts, which is why such methods are not considered natural processes within the meaning of EC 1334/2008.

A combined method that combines a specific alpha-dioxygenase with an aldehyde dehydrogenase and specifically expands the production spectrum is not known. In a recombinant reaction system with two or more enzymes, where the desired catalytic reactions are to take place sequentially and specifically with regard to substrate specificity and selectivity, it is necessary to precisely coordinate the enzymes used so that they are not impaired by endogenous enzymes of the production organism, e.g. in the case of whole-cell catalysis. The reaction control is therefore crucial to ensure that the intermediate products are also recognized by the second or next enzyme in the cascade and are available for it. Such a coordination or combination is always technically demanding and cannot be predicted in a straightforward manner, but rather requires intensive analyses of the enzymes of interest in the respective biotechnological context.

The natural occurrence of some unsaturated aldehydes with one or more double bonds such as 8Z-heptadecenal, 8Z,11Z-heptadecadienal or 8Z,11Z,14Z-heptadecatrienal has been described in pressed juices of cucumber (Cucumber Aroma formation of cucumber (Cucumis sativus L.) and bitter gourd (Momordica charantia L.) by salt-squeezing, Journal of Home Economics of Japan Vol. 60 (2009) No. 10 p. 877-885) or the alga Ulva pertusa (Production of bioflavor by regeneration from protoplasts of Ulvapertusa (Ulvales, Chlorophyta) Hydrobiologia, 1990, Vol. 204, pp 143-149). The content of the aldehydes 8Z-heptadecenal, 8Z,11Z-heptadecadienal, 8Z, 11Z,14Z-heptadecatrienal is given in the literature as 2%, 19.9% and 26.1%.

The odor of 8Z, 11Z-heptadecadienal is described as typical for algae (Production of bioflavor by regeneration from protoplasts of Ulva pertusa (Ulvales, Chlorophyta), Hydrobiologia, 1990, Vol. 204, pp 143-149).

However, the flavor of 4Z,7Z, 10Z,13Z-nonadecatetraenal, 4Z,7Z, 10Z, 13Z, 16Z-nonadecapentaenal or 3,6,9, 12, 15, 18-heneicosahexaenoic acid has not been described.

8Z-pentadecenal is known to be a naturally occurring component of cucumbers (Kemp, A C15 aldehyde from Cucumis sativus, Phytochemistry, Volume 16, Issue 11, 1977, Pages 1831-1832). The taste of 8Z-pentadecenal is mainly described as oily and its flavor-enhancing properties on chicken and grilled beef are also described. Furthermore, a method for the production from 1-nonine and 6-bromo-1-hexanol is known, but this cannot be classified as a natural production process according to EC 1334/2008 (JP 2014043409).

The flavor of 4Z, 7Z, 10Z, 13Z-nonadecatetraenal is not known and no production methods exist.

Various aldehydes can be produced from fatty acids by alpha-oxidation using plant enzymes, as described in WO 2012/025629 A1. The use of algae biomass for the production of unsaturated aldehydes is also known.

Other methods for producing fatty acids from the corresponding oils or fats by enzymatic cleavage are also known from the prior art. In particular, bacterial lipases such as enzymes from Aspergillus niger, Rhizopus oryzae, Penicillium camembertii, Mucor juvanicus, Penicillium roquefortii, porcine pancreas, Candida rugosa, Rhizomucor miehei, Candida antarctica or Rhizops delemar are used for this purpose (Industrial applications of microbial lipases, Enzyme and Microbial Technology Volume 39, Issue 2, Jun. 26, 2006, Pages 235-251).

An object of the present invention was therefore to provide a method for the production of aldehydes or aldehyde mixtures which can be classified as a natural production method according to EC 1334/2008. Furthermore, it was an object to provide an integrated biotechnological process through the targeted coordination of enzymatic metabolic steps, which can provide the products of interest in high yield and purity without intermediate chemical steps.

### SUMMARY OF THE INVENTION

The present invention, according to a primary aspect, thus relates to providing a method for producing saturated and unsaturated aldehydes by means of at least one alpha-dioxygenase having an amino acid sequence according to SEQ ID NO: 1 or 2 or an amino acid sequence having a similarity of 90%, 91%, 92%, 93%, 94%, 95%, 96%, 97%, 98%, 99% or more to SEQ ID NO: 1 or 2, and at least one aldehyde dehydrogenase.

Furthermore, according to one embodiment, the present invention relates to a fermentative method for the production of saturated and unsaturated aldehydes and mixtures thereof. In another embodiment of the method according to the invention, an enzymatic method is provided.

In a further embodiment, the method according to the present invention relates to the use of starting materials which are either of biotechnological, natural or chemical origin.

In yet another embodiment, the method according to the invention relates to preferably used starting materials selected from the group of carboxylic acids and carboxylic acids esterified with alcohols.

A further embodiment relates to the method according to the invention, wherein a carboxylic acid is obtained as a first starting material, which is then used as a modified starting material for a biotechnological method for the production of saturated and unsaturated aldehydes by means of at least one alpha-dioxygenase and at least one aldehyde dehydrogenase.

Furthermore, the method according to the invention relates to preferred product mixtures, microorganisms which are used in the method and amino acid sequences of the enzymes to be preferably used and nucleotide sequences or nucleic acid segments coding for the enzymes to be used.

A further aspect of the present invention relates to the provision of a vector system which encodes the enzymes used according to the invention.

### BRIEF DESCRIPTION OF THE DRAWINGS

FIG. 1 shows the results of the substrate specificity tests in relative activity using different fatty acid substrates.

FIG. 2A shows the result of the whole cell biotransformation of capric acid and myristic using an alpha-dioxygenase according to SEQ ID NO: 2

FIG. 28 shows the result of the whole cell biotransformation of capric acid and myristic using an alpha-dioxygenase according to SEQ ID NO: 1.

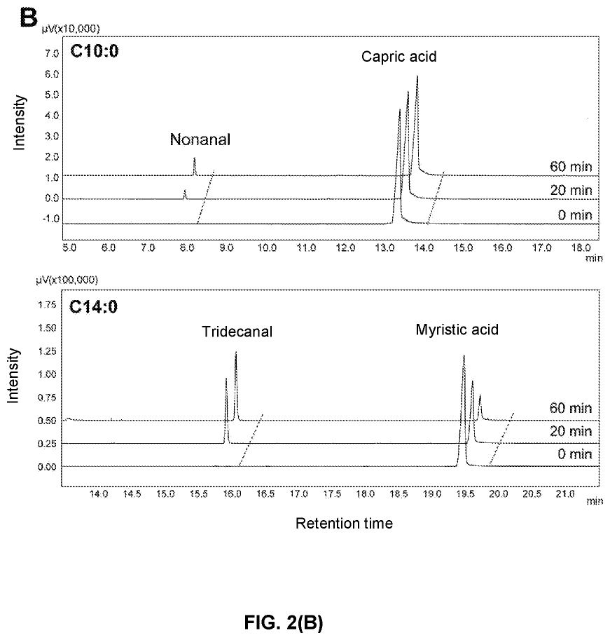

### BRIEF DESCRIPTION OF THE SEQUENCES

SEQ ID NO: 1: Amino acid sequence encoding an alpha-dioxygenase from Leptolyngbya sp. PCC 7375.

SEQ ID NO: 2: Amino acid sequence encoding an alpha-dioxygenase from Calothrix sp. PCC 6303.

SEQ ID NO: 3: Nucleic acid sequence encoding an alpha-dioxygenase from Leptolyngbya sp. PCC 7375.

SEQ ID NO: 4: Nucleic acid sequence encoding an alpha-dioxygenase from Calothrix sp. PCC 6303.

SEQ ID NO: 5: Amino acid sequence encoding an NAD (P) H oxidase from Lactobacillus sanfranciscensis.

SEQ ID NO: 6: Amino acid sequence encoding an NADH oxidase from Lactobacillus brevis.

SEQ ID NO: 7: Nucleic acid sequence encoding an NAD (P) H oxidase from Lactobacillus sanfranciscensis.

SEQ ID NO: 8: Nucleic acid sequence encoding an NADH oxidase from Lactobacillus brevis.

SEQ ID NO: 9: Amino acid sequence encoding for the enzyme VhFALDH from Vibrio harveyi.

SEQ ID NO: 10: Amino acid sequence encoding the 175Q mutant of the enzyme VhFALDH from Vibrio harveyi.

SEQ ID NO: 11: Amino acid sequence encoding the enzyme ReALDH from Rhodococcus erythropolis UPV-1.

SEQ ID NO: 12: Amino acid sequence encoding the enzyme GtALDH from Geobacillus thermodenitrificans.

SEQ ID NO: 13: Amino acid sequence encoding the enzyme Maqu3410, an aldehyde dehydrogenase from Marinobacter aquaeolei VT8.

SEQ ID NO: 14: Amino acid sequence encoding the enzyme Ald1 from Acinetobacter sp. 10 strain M1.

SEQ ID NO: 15: Amino acid sequence encoding the enzyme HFD4, an aldehyde dehydrogenase from Yarrowia lipolytica.

### DEFINITIONS

The term vector system as used herein refers to a system that consists of or contains at least one or more vectors or plasmid vectors. Thus, a vector system may contain a (plasmid) vector encoding several different target genes. Furthermore, a vector system may also comprise a plurality of (plasmid) vectors which in turn contain at least one target gene according to the present disclosure. A vector system may thus comprise only one (plasmid) vector construct or several (plasmid) vector constructs, wherein the latter may be stably or transiently transformed into the corresponding recombinant host organism simultaneously or sequentially, so that the target genes encoded by the individual constructs may be transcribed and translated by the host organism.

The terms amino acid, protein and polypeptide are used interchangeably herein. The amino acids disclosed herein, or functional segments thereof, have enzymatic function when correctly folded. Accordingly, the term enzyme is also used interchangeably herein for the term amino acid.

Whenever the present disclosure refers to sequence homologies or sequence identities of nucleic or protein sequences in terms of percentages, such references are to values as can be calculated using EMBOSS Water Pairwise Sequence Alignments (nucleotides) for nucleic acid sequences or EMBOSS Water Pairwise Sequence Alignments (protein) for amino acid sequences. The tools for local sequence alignments provided by the European Molecular Biology Laboratory (EMBL) European Bioninformatics Institute (EBI) use a modified Smith-Waterman algorithm. Furthermore, when performing the respective pairwise alignment of two sequences using the modified Smith-Waterman algorithm, reference is made to the default parameters currently specified by the EMBL-EBI. These are (i) for amino acid sequences: Matrix=BLOSUM62, Gap open penalty=10 and Gap extend penalty=0.5 and (ii) for nucleic acid sequences: Matrix=DNAfull, Gap open penalty=10 and Gap extend penalty=0.5. The calculation of a sequence homology or sequence identity must always be calculated using the total length of a sequence.

The growing and culturing, isolation and purification of a recombinant microorganism or fungus or a protein or enzyme encoded by a nucleic acid according to the disclosure of the present invention is known to the skilled person in the field.

The terms protein, polypeptide and enzyme are used interchangeably due to the always enzymatic function of the gene products disclosed herein. Similarly, the terms gene and nucleic acid (segment) are used interchangeably for the purposes of the present disclosure.

The nucleic acids, nucleotide sequences or nucleic acid segments (these terms are used interchangeably for DNA sequences encoding a functional enzyme, or a functional region thereof) used to express a desired target protein according to the present invention may be codon-optimized, if desired, i.e. the codon usage of a gene is adapted to that of the recombinant microorganism or fungus chosen as the expression strain. It is known to the skilled person in the field that a desired target gene encoding a protein of interest can be modified without changing the translated protein sequence to accommodate the specific species-dependent codon usage. Thus, the nucleic acids of the present invention to be transformed can be specifically adapted to the codon usage of E. coli or another bacterium, Saccharomyces spp. or another yeast.

The disclosed carboxylic acids can be present either in their free form or esterified to alcohols, which occur, for example, as components of lipids.

Unless the configuration(s) of (Z)/(E) isomers is specifically indicated herein for a named compound, the indication includes all possible configuration isomers of the corresponding compound, or a mixture thereof. It should be noted that certain enzymes disclosed herein are capable of recognizing and converting both isomers of a compound of interest.

### DETAILED DESCRIPTION

The problem of the present invention is solved primarily by providing a biotechnological method for producing at least one unsaturated aldehyde and its corresponding carboxylic acid and/or at least one saturated aldehyde and its corresponding carboxylic acid, wherein the method comprises providing at least one alpha-dioxygenase having an amino acid sequence according to SEQ ID NO: 1 or 2, or an amino acid sequence having a similarity of 90%, 91%, 92%, 93%, 94%, 95%, 96%, 97%, 98%, 99% or more to SEQ ID NO: 1 or 2, and at least one aldehyde dehydrogenase.

The aldehyde obtained is always shortened by one carbon atom compared to the starting material used. In the context of the present invention, a saturated aldehyde refers to an aldehyde without double bonds, wherein an unsaturated aldehyde refers to aldehydes with double bonds present. The corresponding carboxylic acid is a compound with the same chain length and the same degree of saturation as the aldehyde obtained, but which carries at least one carboxyl group. The alpha-dioxygenase used according to the invention according to SEQ ID NO: 1 or 2, or an amino acid sequence with a similarity of 90%, 91%, 92%, 93%, 94%, 95%, 96%, 97%, 98%, 99% or more to SEQ ID NO: 1 or 2, catalyzes the oxidation of carboxylic acids of chain length C6-C22 at the alpha-carbon atom, so that the aldehyde corresponding to the carboxylic acid and shortened by one carbon atom can be formed.

The alpha-dioxygenase according to SEQ ID NO: 1 or 2 to be used according to the invention have the advantage that it has a particularly advantageous substrate and product spectrum. The alpha-dioxygenases according to the invention from Leptolyngbya sp. (SEQ ID NO: 1) and Calothrix parientina (SEQ ID NO: 2) have a preferential activity towards short fatty acids with 10 to 14 carbon atoms. At the same time, the substrate spectrum is not very narrow, so that these enzyme variants can be used particularly advantageously in the methods disclosed herein.

Due to its substrate specificity, the at least one aldehyde dehydrogenase used catalyzes the oxidation of an aldehyde to the corresponding carboxylic acid, which in turn can be further converted to an aldehyde shortened by one carbon atom using the alpha-dioxygenase. The method according to the invention can thus be used to specifically produce aldehyde mixtures with aldehydes that have different chain lengths.

In one embodiment of the method according to the invention, the alpha-oxidation and the aldehyde oxidation can be carried out simultaneously, wherein in a further embodiment the alpha-oxidation can be carried out first and then the aldehyde oxidation, wherein the reaction cycle can be continued in both embodiments until the desired product is obtained.

It is particularly preferred in the context of the present invention if the fatty acids obtained by alpha-oxidation are used as preferred substrates for the aldehyde oxidation.

In a preferred embodiment of the method according to the invention, the biotechnological production method can be a fermentative method comprising the following steps:
i. Providing at least one recombinant microorganism or fungus containing a nucleic acid segment comprising at least one gene coding for an alpha-dioxygenase and/or at least one gene coding for an aldehyde dehydrogenase having a nucleic acid sequence according to one of SEQ ID NO: 3 or 4, or a nucleic acid sequence having a similarity of 90%, 91%, 92%, 93%, 94%, 95%, 96%, 97%, 98%, 99% or more to one of SEQ ID NO: 3 or 4;
ii. Cultivating the at least one recombinant microorganism under conditions which permit the expression of the corresponding expression product;
iii. Adding at least one carboxylic acid or at least one carboxylic acid esterified with an alcohol to the at least one cultured recombinant microorganism;
iv. Obtaining at least one unsaturated aldehyde and its corresponding carboxylic acid and/or at least one saturated aldehyde and its corresponding carboxylic acid by reaction of the at least one alpha-dioxygenase and the at least one aldehyde dehydrogenase with the at least one carboxylic acid or the carboxylic acid esterified to an alcohol from step iii.

A fermentative method in the context of the present invention refers to a method in which, in at least one step, a recombinant microorganism is cultivated which expresses the at least one alpha-dioxygenase and the at least one aldehyde dehydrogenase required to produce the product according to the invention. The expression product here is the at least one alpha-dioxygenase according to SEQ ID NO: 1 or 2 or an amino acid sequence with a similarity of 90%, 91%, 92%, 93%, 94%, 95%, 96%, 97%, 98%, 99% or more to one of SEQ ID NO: 1 or 2, and/or the at least one aldehyde dehydrogenase. According to one embodiment, the conversion of the starting material can take place by secretion of the enzyme into the reaction mixture in which the starting materials are present, whereas according to a further embodiment, the conversion can take place in the cytosol of the at least one microorganism and the product can subsequently be secreted.

In yet another preferred embodiment, the fermentative method may be a biotransformation with quiescent cells and/or microorganisms comprising an additional step (ii.a) comprising removing the cultivation medium and incorporating the cultured recombinant microorganism into an aqueous buffer. The expression products present in the cells catalyze the reaction of the at least one carboxylic acid or at least one carboxylic acid esterified with an alcohol added in step iii. to obtain in step iv. at least one unsaturated aldehyde and its corresponding carboxylic acid and/or at least one saturated aldehyde and its corresponding carboxylic acid.

Providing in the context of the present invention always means involving an active step to make available a starting material, an enzyme or the like. According to one embodiment, the providing can always be a technical step, such as the cloning of a reaction organism or the production of a starting material.

In a further preferred embodiment of the method according to the invention, the biotechnological production method may be an enzymatic method comprising the following steps:
i. Providing at least one alpha-dioxygenase having an amino acid sequence according to SEQ ID NO: 1 or 2 or an amino acid sequence with a similarity of 90%, 91%, 92%, 93%, 94%, 95%, 96%, 97%, 98%, 99% or more to one of SEQ ID NO: 1 or 2, and at least one aldehyde dehydrogenase, wherein the at least one alpha-dioxygenase and/or the at least one aldehyde dehydrogenase can be produced naturally, chemically or biotechnologically;
ii. Adding at least one carboxylic acid or at least one carboxylic acid esterified with an alcohol to the enzymes provided in step i;
iii. Obtaining at least one unsaturated aldehyde and its corresponding carboxylic acid and/or at least one saturated aldehyde and its corresponding carboxylic acid by reaction of the at least one alpha-dioxygenase and the at least one aldehyde dehydrogenase with the at least one carboxylic acid or the carboxylic acid esterified to an alcohol from step ii.

An enzymatic method in the context of the present invention refers to a method in which the at least one alpha-dioxygenase and the at least one aldehyde dehydrogenase may be present purified or partially purified outside a microorganism. In one embodiment, the two enzymes may be present as a cell lysate, which denotes the state of microorganisms which can no longer be cultivated and which have been digested by physical, chemical or enzymatic processes. In a further embodiment, the enzymes may be present with a purity of>80%, having been purified by a purification method known to the skilled person. In yet another embodiment, the enzymes may be present with a purity<80%, wherein they have been partially purified by a purification method known to a person skilled in the art. In still another embodiment, the enzymes may be obtained by protein synthesis and optionally purified.

A starting material of biotechnological origin is a starting material produced by fermentation of microorganisms, whereby a starting material of natural origin can be obtained from a natural source, for example a plant, a fungus, an animal, a microorganism or from the soil. A starting material of chemical origin can be produced by a chemical synthesis method from precursors of the starting material.

In a further preferred embodiment, at least one of the starting materials of the method according to the invention can be of biotechnological, natural or chemical origin. The foregoing applies to the starting materials of biotechnological, natural or chemical origin.

According to a further preferred embodiment of the method according to the invention, the starting material can be selected from the group consisting of fully saturated, mono-, di-or tri-unsaturated or mono-branched fatty acids from C6 to C20, preferably from C8 to C18, particularly preferably from the group consisting of caprylic acid, capric acid, 4-ethyloctanoic acid, lauric acid, 10-methylundecanoic acid, 9-methylundecanoic acid, 10-methyldodecanoic acid, 11-methyldodecanoic acid, 11-methyltridecanoic acid, 12-methyltridecanoic acid, 13-methyltetradecanoic acid, 12-methyltetradecanoic acid, 13-methylpentadecanoic acid, 14-methylpentadecanoic acid, 14-methylhexadecanoic acid, 15-methylhexadecanoic acid, myristic acid, palmitic acid, stearic acid, palmitoleic acid, petroselinic acid, margaric acid, cis-vaccenic acid, linoleic acid, stearidonic acid, gamma-linolenic acid, alpha-linolenic acid, oleic acid, punicic acid, alpha-elaeostearic acid and the underlying fatty acid esters, in particular the fatty oils selected from olive oil, low-erucic acid rapeseed oil, macadamia nut oil, sea buckthorn pulp oil, borage oil, black currant seed oil, parsley seed oil, dill seed oil, pomegranate seed oil, coconut oil, sunflower oil, wheat germ oil, rice germ oil, peanut oil, sesame oil, palm fruit oil, grape seed oil and mushroom oils obtained from any of the genera selected from Conidiobolus, Flammulina, Fomes, Ganoderma, Mortierella, Panellus, Pleurotus, Psathyrella, Stereum, Umbelopsis and derivatives thereof. the carboxylic acids and the carboxylic acids esterified with alcohols, preferably wherein the starting materials are selected from the group consisting of palmitoleic acid, arachidonic acid, alpha-linolenic acid, oleic acid, punicic acid, alpha-elaeosteraric acid, docosahexaenoic acid, eicosapentaenoic acid, petroselinic acid, chaulmoograic acid, alpha-licaric acid, olive oil, rapeseed oil, macadamia nut oil, sea buckthorn pulp oil, tung oil, fish oil, borage oil, chaulmoogra oil, parsley oil, oiticica oil, pomegranate seed oil, coconut oil, sunflower oil, grape seed oil and mushroom oils obtained from any of the genera selected from Conidiobolus, Flammulina, Fomes, Ganoderma, Mortierella, Panellus, Pleurotus, Psathyrella, Stereum, Umbelopsis and derivatives thereof.

In a further preferred embodiment, the method may comprise the further steps of:
(a) obtaining at least one carboxylic acid, preferably at least one carboxylic acid as defined herein, preferably comprising an odd number of carbon atoms, as product;
(b) optionally: purifying the at least one carboxylic acid obtained as product;
(c) using the at least one carboxylic acid obtained as product as modified starting material for carrying out a biotechnological method for producing at least one unsaturated aldehyde and its corresponding carboxylic acid and/or at least one saturated aldehyde and its corresponding carboxylic acid, wherein the method comprises providing at least one alpha-dioxygenase and at least one aldehyde dehydrogenase, preferably wherein the method is a method according to the present invention.

A “modified starting material” in the context of the present invention, is a starting material which is not the same starting material as the fatty or carboxylic acid to be used in the method according to the invention. This modified starting material may be the product of a previously performed enzymatic reaction with an alpha-dioxygenase and an aldehyde dehydrogenase as described herein.

In a preferred embodiment, the method according to the present invention can be carried out as a cycle reaction. This means that carboxylic acids, preferably fatty acids, with an odd number of carbon atoms, such as those that can be obtained as a product by a method described above, are used again as a starting material in an integrated process, wherein at least one of the fatty acids of the method according to the invention is of biotechnological, natural or chemical origin. The above applies to the starting materials of biotechnological, natural or chemical origin.

In a further preferred embodiment, the product of the method according to the invention can be at least one saturated aldehyde and its corresponding carboxylic acid and/or an unsaturated aldehyde and its corresponding carboxylic acid. Hereby, the products of the method according to the invention can preferably be selected from the group comprising 7Z, 10Z-hexadecadienal, 8Z, 11Z-heptadecadienal, 6E,9E-pentadecadienal, 7Z-hexadecenal, 8Z-pentadecenal, 7Z-tetradecenal, 8Z, 11Z, 14Z-heptadecatrienal, 7Z, 10Z, 13Z-hexadecatrienal, 4Z,7Z, 10Z, 13Z-nonadecatetraenal, 8Z, 10E, 12E-heptadecatrienal, 8Z, 10E, 12Z-heptadecatrienal, 7Z,9E, 11Z-hexadecatrienal, 7Z,9E, 11E-hexadecatrienal, 9-decenal, 10-undecenal, 3Z-decenal, 4Z-undecenal, 7Z-dodecenal, 8Z-tridecenal, 7Z-tetradecenal, 8Z-pentadecenal, 9Z-tetradecenal, 10Z-pentadecenal, 7Z-pentadecenal, 8Z-hexadecenal, 9Z-hexadecenal, 10Z-heptadecenal, 5Z,8Z-tetradecadienal, 6Z,9Z-pentadecadienal, 7Z, 10Z-tetradecadienal, 8Z, 11Z-pentadecadienal, 7Z,9E-hexadecadienal, 8Z, 10E-heptadecadienal, 8E, 10Z-hexadecadienal, 9E, 11Z-heptadecadienal, 9-methylundecanal, 10-methylundecanal-, 10-methyldodecanal, 11-methyldodecanal, 11-methyltridecanal, 12-methyltridecanal-, 12-methyltetradecanal, 13-methyltetradecanal and 13-methylpentadecanal.

In the context of the present invention, all compounds in which the positions of the double bond are not specifically indicated with respect to the (Z)/(E) configuration include all possible positions of the double bond and mixtures of the relevant compounds.

According to a further embodiment of the method according to the invention, the at least one recombinant microorganism may be selected from the group consisting of Escherichia coli, preferably E. coli BL21, E. coli MG1655, E. coli W3110 and their derivatives, Bacillus spp, preferably B. licheniformis, B. subitilis or B. amyloliquefaciens, and their derivatives, Saccharomyces spp, preferably S. cerevesiae, and their derivatives, Hansenula or Komagatella spp, preferably K. phaffii and H. polymorpha, and their derivatives, Kluyveromyces spp, preferably K. lactis.

According to a preferred embodiment, at least one NADH oxidase and/or at least one lipase may additionally be provided.

NADH oxidases are enzymes that can catalyze the oxidation of an aldehyde due to their substrate specificity and regioselectivity. In a preferred embodiment of the method according to the invention, these can be used as additional enzymes in order to make the closely coordinated methods even more efficient by recycling the corresponding co-factor.

In another preferred embodiment of the method according to the invention, the at least one NADH oxidase may comprise an amino acid sequence, wherein the amino acid sequence is independently selected from the group consisting of SEQ ID NOs: 5 and 6, or a sequence having at least 90%, 91%, 92%, 93%, 94%, 95%, 96%, 97%, 98%, 99% or 100% sequence identity thereto. In an embodiment, the amino acid sequence encoding the NADH oxidase is encoded by a nucleic acid sequence selected from the group consisting of SEQ ID NOS: 7 and 8, or a sequence having at least 90%, 91%, 92%, 93%, 94%, 95%, 96%, 97%, 98%, 99% or 100% sequence identity thereto.

Moreover, an enzyme catalyzing the reaction according to the various aspects and embodiments of the present invention may be a catalytically active domain or fragment of the particular enzyme from which it is derived.

In a further preferred embodiment, the at least one lipase may be a commercial lipase selected from the group consisting of Candida antarctica, Aspergillus niger, Rhizopus oryzae, Penicillium camembertii, Mucor juvanicus, Penicillium roqueforti, porcine pancreas, Candida rugosa, Rhizomucor miehei, Candida antarctica or Rhizopus delemar.

In a further preferred embodiment, the at least one aldehyde dehydrogenase may comprise or consist of an amino acid sequence, wherein the amino acid sequence may be selected from the group consisting of SEQ ID NOs: 9, 10, 11, 12, 13, 14 or 15 or a sequence having at least 90%, 91%, 92%, 93%, 94%, 95%, 96%, 97%, 98%, 99% or 100% sequence identity thereto.

A further embodiment of the present invention may be directed to nucleic acid sequences or segments thereof encoding polypeptides having enzymatic function for the purpose of purification, secretion, detection or localization of the at least one alpha-dioxygenase and/or the at least one aldehyde dehydrogenase and/or the at least one lipase and/or the at least one NADH oxidase. These nucleic acid segments are also referred to as tag sequences and may precede (N-terminal) and/or follow (C-terminal) the nucleic acid segments encoding the at least one alpha-dioxygenase and/or the at least one aldehyde dehydrogenase. Preferred tag sequences are selected from the following list: Polyhistidine (His) tag, glutathione S-transferase (GST) tag, thioredoxin tag, FLAG tag, green fluorescent protein (GFP) tag, streptavidin tag, maltose binding protein (MBP) tag, chloroplast transit peptide, mitochondrial transit peptide and/or secretion tag. Moreover, the skilled person is aware of a number of other suitable tag sequences for microorganisms or fungi of interest as a production strain, wherein these tag sequences allow the secretion of a polypeptide of interest directly into the culture supernatant. This may be advantageous in some embodiments to more easily obtain and optionally purify a polypeptide of interest.

In a further aspect of the present invention, the invention relates to a vector system, preferably a plasmid vector system, comprising at least one vector or plasmid vector comprising a nucleic acid segment (a) comprising at least one gene coding for an alpha-dioxygenase having a nucleic acid sequence according to one of SEQ ID NO: 3 or 4 or a nucleic acid sequence having a similarity of 90%, 91%, 92%, 93%, 94%, 95%, 96%, 97%, 98%, 99% or more to one of SEQ ID NO: 3 or 4, and a nucleic acid segment (b) comprising at least one gene encoding an aldehyde dehydrogenase; and optionally comprising, as part of the nucleic acid portion (a) and/or (b), a nucleic acid portion comprising at least one gene encoding an NADH oxidase and/or a nucleic acid portion comprising at least one gene encoding a lipase, wherein the nucleic acid portion (a) and/or the nucleic acid portion (b) is provided on the same vector, or on two or more separate vectors.

In a further aspect, the present invention relates to a composition comprising an aldehyde mixture obtained by a method according to the invention, characterized in that the composition comprises at least one or preferably at least two or three aldehydes selected from the group consisting of 7Z-tetradecenal, 8Z-pentadecenal, pentadecanal, 7Z-hexadecenal and 8Z-heptadecenal, wherein the one or more aldehyde(s) are each present in a proportion of about 0.0001-20 wt. %, preferably 0.001-10% wt. %, more preferably 0.01-5 wt. %, each with respect to the total composition, and wherein the composition optionally contains further aldehydes obtained by a method according to any one of claims 1 to 12, or wherein the composition comprises 7Z-tetradecenal in a proportion of about 0.0001-20 wt. %, or wherein the composition comprises 8Z-pentadecenal in a proportion of about 0.0001-20 wt. %, with respect to the total composition, or wherein the composition comprises pentadecanal in an amount of about 0.0001-20 wt. %, with respect to the total composition, or wherein the composition comprises 7Z-hexadecenal in an amount of about 0.0001-20 wt. %, with respect to the total composition, or wherein the composition comprises 8Z-heptadecenal in an amount of about 0.0001-20 wt. %, with respect to the total composition, or wherein the composition comprises 8Z-heptadecenal in an amount of about 0.0001-20 wt. %, with respect to the total composition, or wherein the composition comprises at least 8Z-pentadecenal and pentadecanal, wherein the proportion of 8Z-pentadecenal and pentadecanal in the total composition comprises about 0.0001-20 wt. %, or wherein the composition comprises at least 8Z-pentadecenal and 8Z-heptadecenal, wherein the proportion of 8Z-pentadecenal and 8Z-heptadecenal in the total composition comprises about 0.0001-20 wt. %.

A composition in the context of the present invention is a semi-finished or finished product composition and is preferably selected from the group consisting of a preparation for nutrition or enjoyment or a cosmetic or cleansing preparation.

Preparations for nutrition or enjoyment within the meaning of the present invention are, for example, bakery products (e.g. bread, dry cookies, cakes, other baked goods), confectionery (e.g. chocolates, chocolate bar products, other bar products, fruit gums, hard and soft caramels, chewing gum), alcoholic or non-alcoholic beverages (e.g. coffee, tea, wine, wine-containing drinks, beer, beer-containing drinks, liqueurs, schnapps, brandies, fruit-based soft drinks, isotonic drinks, soft drinks, nectars, fruit and vegetable juices, fruit or vegetable juice preparations), instant drinks (e.g., instant cocoa drinks, instant tea drinks, instant coffee drinks, instant fruit drinks), meat products (e.g. ham, fresh or raw sausage preparations, seasoned or marinated fresh or cured meat products), eggs or egg products (dried eggs, egg whites, egg yolks), cereal products (e.g. breakfast cereals, muesli bars, pre-cooked ready-to-eat rice products), dairy products (e.g. milk drinks, buttermilk drinks, milk ice cream, yoghurt, kefir, cream cheese, soft cheese, hard cheese, dried milk powder, whey, butter, buttermilk, partially or fully hydrolyzed milk protein-containing products), products made from soy protein or other soybean fractions (e.g. soy milk and products made from it, fruit drinks with soy protein, preparations containing soy lecithin, fermented products such as tofu or tempeh or products made from them), fruit preparations (e.g. jams, fruit ice cream, fruit sauces, fruit fillings), vegetable preparations (e.g. ketchup, sauces, dried vegetables, frozen vegetables, pre-cooked vegetables, boiled vegetables), snacks (e.g. baked or deep-fried potato potato chips or potato dough products, corn or peanut-based extrudates), fat and oil-based products or emulsions thereof (e.g. mayonnaise, remoulade, dressings), other ready meals and soups (e.g. dry soups, instant soups, pre-cooked soups), spices, seasoning mixtures and, in particular, seasonings which are used, for example, in the snack sector. The preparations within the meaning of the invention can also be used as semi-finished products for the production of other preparations for nutrition or enjoyment. The preparations within the meaning of the invention can also be in the form of capsules, tablets (uncoated and coated tablets, e.g. enteric coatings), dragees, granules, pellets, solid mixtures, dispersions in liquid phases, as emulsions, as powders, as solutions, as pastes or as other preparations that can be swallowed or chewed as food supplements.

A cosmetic or cleansing preparation within the meaning of the present invention is a preparation which can be used for cleaning, caring for or perfuming the body or for cleansing. Preferred cosmetic preparations are selected from the group consisting of floor cleaners, window glass cleaners, dishwashing detergents, bathroom and sanitary cleaners, scouring milk, solid and liquid toilet cleaners, powdered and foamed carpet cleaners, textile fresheners, ironing aids, liquid detergents, powdered detergents, laundry pretreatment agents such as bleaching agents, softeners and stain removers, laundry softeners, laundry soaps, washing tablets, disinfectants, surface disinfectants and air fresheners in liquid, gel or solid carrier applied form, aerosol sprays, waxes and polishes such as furniture polishes, floor waxes, shoe polishes and personal care products such as solid and liquid soaps, shower gels, shampoos, shaving soaps, shaving foams, bath oils, cosmetic emulsions of the oil-in-water, water-in-oil and water-in-oil-in-water type, e.g. skin creams and lotions, face creams and lotions, sun protection creams and lotions, after-sun creams and lotions, hand creams and lotions, foot creams and lotions, depilatory creams and lotions, after-shave creams and lotions, tanning creams and lotions, hair care products such as hair sprays, hair gels, strengthening hair lotions, hair conditioners, permanent and semi-permanent hair dyes, hair styling products such as cold waves and hair straightening products, hair washes, hair creams and lotions, deodorants and antiperspirants such as underarm sprays, roll-on sprays, deodorant sticks, deodorant creams, decorative cosmetic products such as eye shadow, nail polish, make-up, lipsticks, mascara, candles, lamp oils, incense sticks, insecticides, repellents and fuels.

In a preferred embodiment, depending on the embodiment of the method according to the invention, the composition obtained contains further aldehydes, carboxylic acids or alcohols selected from the group consisting of dodecanol, tridecanal, tridecanol, tetradecanal, 8Z-tetradecenol, tetradecanol, pentadecanal, pentadecanol, octanoic acid, decanoic acid, tridecanal, tridecenoic acid, pentadecenal and 8Z-tetradecenoic acid.

In a further embodiment, the individual components of the resulting mixtures can be partially or completely purified using a method known to the skilled person. A major advantage of these mixtures is therefore their universal applicability in a large number of industrial sectors, since they can be used both as a mixture and as individual components.

In the following, the present invention is further explained with reference to non-limiting examples and based on the disclosure provided in the sequence listing and figures.

### EXAMPLES

### Example 1: Expression of alpha-dioxygenases According to SEQ ID NO: 1 or 2

The nucleotide sequences according to SEQ ID NO: 3 (LepDOX) and 4 (CalDOX) were cloned into the expression vector pET28a (Novagen, Darmstadt, Germany). The obtained vectors were transformed into competent E. coli BL21 (DE3) cells. E. coli cells containing the α-DOX genes after selection were cultured in LB media with 50 μg/mL kanamycin. Protein expression was induced by adding 0.5 mM isopropyl-B-D-1-thiogalactopyranoside (IPTG), after which the cells were incubated at 18° C. for 18 hours and harvested by centrifugation at 4,500 g and 4° C. for 20 minutes.

The cell pellets were then resuspended in buffer (50 mM Tris-HCl, 200 mM NaCl and 20 mM imidazole; pH 8.0) and digested using an ultrasonicator. During cell lysis for the α-DOX, 1% (v/v) Triton X-100 was added to facilitate extraction. The total cell lysate was centrifuged twice at 10,000×g and 4° C. for 30 min, and the supernatant containing the soluble crude extract was applied to a column of Ni agarose resin (Carl Roth, Karlsruhe, Germany) and purified by immobilized metal ion affinity chromatography (IMAC). The His-tagged proteins were eluted by addition of buffer B (50 mM Tris-HCl, 200 mM NaCl and 300 mM imidazole; pH 8.0). The proteins were analyzed by sodium dodecyl sulfate polyacrylamide gel electrophoresis (SDS-PAGE) to verify their expression and purity. Protein concentrations were quantified with the Bradford assay using bovine serum albumin (BSA) as a standard (Carl Roth).

### Example 2: Substrate Spectrum of alpha-dioxygenases According to SEQ ID NO: 1 or 2

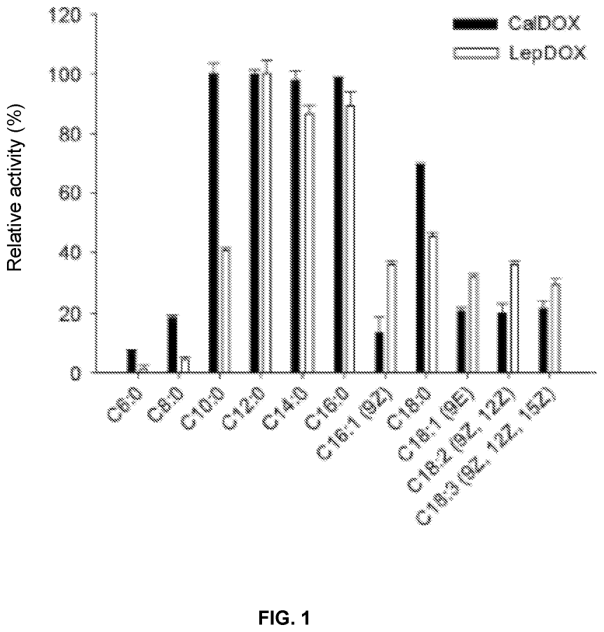

The two α-DOX according to SEQ ID NO: 1 (LepDOX) or SEQ ID NO: 2 (CalDOX) were incubated with various fatty acids with a chain length of 6 carbon atoms to 18 carbon atoms and showed maximum relative activity for lauric acid (C12), myristic acid (C14) and palmitic acid (C16). Further good activity was observed for short-chain fatty acids (C8 to C10) (FIG. 1).

### Example 3: Whole Cell Biotransformation

Expression of the recombinant CalDOX and LepDOX enzymes was induced in E. coli cells as in Example 1. After harvesting the cells by centrifugation at 9,600×g and 4° C. for 10 minutes, the cells were washed with medium containing 200 mM potassium phosphate (pH 7.4) containing 50 mM NaCl and then centrifuged under the same conditions. After decanting the supernatant, the cells were resuspended in the same medium.

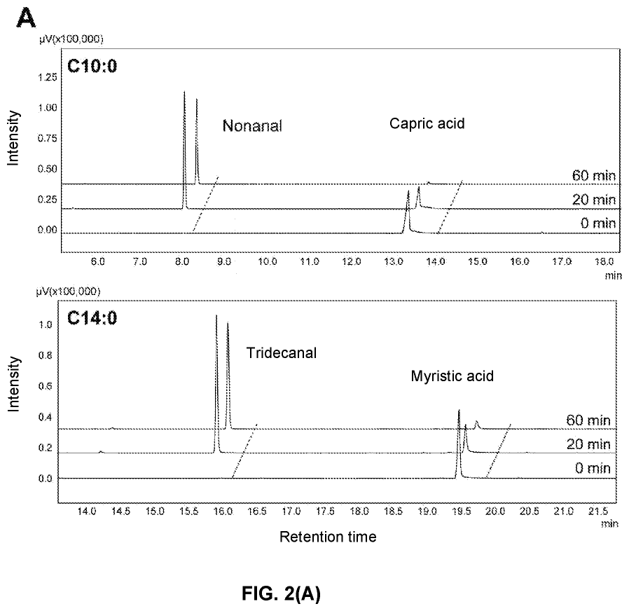

Whole-cell biotransformations were performed with E. coli cells at 12.5 g wet weight in buffer at 35° C. and 1,000 rpm with a reaction volume of 300 μL using a ThermoMixer (Eppendorf, Hamburg, Germany). The reaction was initiated by adding 15 μL of 100 mM fatty acid substrates (capric acid or myristic acid) from a stock DMSO solution to 285 μL of the quiescent cell suspension. After incubation for the indicated times, biotransformation was stopped by adding 30 μl of 2 M HCl, and the samples were extracted three times with an equal volume of ethyl acetate and dried over anhydrous Na2SO4 and analyzed. Conversion of both fatty acids was observed within 20 (C10) or 40 (C14) minutes (FIG. 2).

The intermediate product obtained can then be converted to the end product using an aldehyde dehydrogenase to obtain an aldehyde and its corresponding carboxylic acid.

1-13. (canceled)

14. A biotechnological method for producing at least one unsaturated aldehyde and its corresponding carboxylic acid and/or at least one saturated aldehyde and its corresponding carboxylic acid, wherein the method comprises providing at least one alpha-dioxygenase having an amino acid sequence according to SEQ ID NO: 1 or SEQ ID NO: 2, or an amino acid sequence having a similarity of 90% or more to SEQ ID NO: 1 or SEQ ID NO: 2, and at least one aldehyde dehydrogenase.

15. The method according to claim 14, wherein the biotechnological method is a fermentative method comprising:
(i) providing at least one recombinant microorganism or fungus comprising a nucleic acid segment comprising at least one gene coding for an aldehyde dehydrogenase and/or at least one gene coding for an alpha-dioxygenase having a nucleic acid sequence according to SEQ ID NO: 3 or SEQ ID NO: 4 or a nucleic acid sequence having a similarity of 90% or more to SEQ ID NO: 3 or SEQ ID NO: 4;
(ii) cultivating the at least one recombinant microorganism under conditions which permit the expression of a corresponding expression product;
(iii) adding at least one carboxylic acid or at least one carboxylic acid esterified with an alcohol as a starting material to the at least one cultivated, recombinant microorganism;
(iv) obtaining the at least one unsaturated aldehyde and its corresponding carboxylic acid and/or the at least one saturated aldehyde and its corresponding carboxylic acid by reaction of the at least one alpha-dioxygenase and the at least one aldehyde dehydrogenase with the at least one carboxylic acid or the carboxylic acid esterified with an alcohol.

16. The method according to claim 14, wherein the biotechnological method is an enzymatic method comprising:
(i) providing at least one alpha-dioxygenase having an amino acid sequence according to SEQ ID NO: 1 or SEQ ID NO: 2 or an amino acid sequence with a similarity of 90% or more to SEQ ID NO: 1 or SEQ ID NO: 2, and at least one aldehyde dehydrogenase, wherein the at least one alpha-dioxygenase and/or the at least one aldehyde dehydrogenase can be produced naturally, chemically, or biotechnologically;
(ii) adding at least one carboxylic acid or at least one carboxylic acid esterified with an alcohol;
(ii) obtaining the at least one unsaturated aldehyde and its corresponding carboxylic acid and/or the at least one saturated aldehyde and its corresponding carboxylic acid by reaction of the at least one alpha-dioxygenase and the at least one aldehyde dehydrogenase with the at least one carboxylic acid or the at least one carboxylic acid esterified with an alcohol.

17. The method according to claim 14, wherein one or more starting materials are of biotechnological origin, natural origin, chemical origin, or combinations thereof.

18. The method according to claim 14, wherein one or more starting materials are selected from fully saturated, mono-, di-, or tri-unsaturated or mono-branched C6 to C20 fatty acids.

19. The method according to claim 18, wherein the one or more starting materials are selected from caprylic acid, capric acid, 4-ethyloctanoic acid, lauric acid, 10-methylundecanoic acid, 9-methylundecanoic acid, 10-methyldodecanoic acid, 11-methyldodecanoic acid, 11-methyltridecanoic acid, 12-methyltridecanoic acid, 13-methyltetradecanoic acid, 12-methyltetradecanoic acid, 13-methylpentadecanoic acid, 14-methylpentadecanoic acid, 14-methylhexadecanoic acid, 15-methylhexadecanoic acid, myristic acid, palmitic acid, stearic acid, palmitoleic acid, petroselinic acid, margaric acid, cis-vaccenic acid, linoleic acid, stearidonic acid, gamma-linolenic acid, alpha-linolenic acid, oleic acid, punicic acid, alpha-elaeostearic acid, or fatty acid esters thereof.

20. The method according to claim 18, wherein the one or more starting materials are selected from olive oil, rapeseed oil low in erucic acid, macadamia nut oil, sea buckthorn pulp oil, borage oil, black currant seed oil, parsley seed oil, dill seed oil, pomegranate seed oil, coconut oil, sunflower oil, wheat germ oil, rice germ oil, peanut oil, sesame oil, palm fruit oil, grape seed oil, mushroom oils obtained from any of the genera selected from Conidiobolus, Flammulina, Fomes, Ganoderma, Mortierella, Panellus, Pleurotus, Psathyrella, Stereum, Umbelopsis, or their derivatives of carboxylic acids and carboxylic acids esterified with alcohols.

21. The method according to claim 20, wherein the one or more starting materials are selected from palmitoleic acid, arachidonic acid, alpha-linolenic acid oleic acid, punicic acid, alpha-elaeosteraric acid, docosahexaenoic acid, eicosapentaenoic acid, petroselinic acid, chaulmoograic acid, alpha-licaric acid, olive oil, rapeseed oil, macadamia nut oil, sea buckthorn pulp oil, tung oil, fish oil, borage oil, chaulmoogra oil, parsley oil, oiticica oil, pomegranate seed oil, coconut oil, sunflower oil, grape seed oil, mushroom oils obtained from any of the genera selected from Conidiobolus, Flammulina, Fomes, Ganoderma, Mortierella, Panellus, Pleurotus, Psathyrella, Stereum, or Umbelopsis, or derivatives thereof.

22. The method according to claim 14, wherein a product of the method is at least one saturated aldehyde and its corresponding carboxylic acid and/or at least one unsaturated aldehyde and its corresponding carboxylic acid selected from 7Z, 10Z-hexadecadienal, 8Z, 11Z-heptadecadienal, 6E,9E-pentadecadienal, 7Z-hexadecenal, 8Z-pentadecenal, 8Z, 11Z, 14Z-heptadecatrienal, 7Z, 10Z, 13Z-hexadecatrienal, 4Z,7Z, 10Z, 13Z-nonadecatetraenal, 8Z, 10E, 12E-heptadecatrienal, 8Z, 10E, 12Z-heptadecatrienal, 7Z,9E,11Z-hexadecatrienal, 7Z,9E, 11E-hexadecatrienal, 9-decenal, 10-undecenal, 3Z-decenal, 4Z-undecenal, 7Z-dodecenal, 8Z-tridecenal, 7Z-tetradecenal, 8Z-pentadecenal, 9Z-tetradecenal, 10Z-pentadecenal, 7Z-pentadecenal, 8Z-hexadecenal, 9Z-hexadecenal, 10Z-heptadecenal, 5Z,8Z-tetradecadienal, 6Z,9Z-pentadecadienal, 7Z, 10Z-tetradecadienal, 8Z, 11Z-pentadecadienal, 7Z,9E-hexadecadienal, 8Z, 10E-heptadecadienal, 8E, 10Z-hexadecadienal, 9E, 11Z-heptadecadienal, 9-methylundecanal, 10-methylundecanal, 10-methyldodecanal, 11-methyldodecanal, 11-methyltridecanal, 12-methyltridecanal, 12-methyltetradecanal, 13-methyltetradecanal, or 13-methylpentadecanal.

23. The method according to claim 14, further comprising:
(a) obtaining at least one carboxylic acid;
(b) optionally, purifying the at least one carboxylic acid;
(c) employing the at least one carboxylic acid as modified starting material for carrying out a biotechnological method for producing at least one unsaturated aldehyde and its corresponding carboxylic acid and/or at least one saturated aldehyde and its corresponding carboxylic acid, wherein the method comprises providing at least one alpha-dioxygenase and at least one aldehyde dehydrogenase.

24. The method according to claim 15, wherein the at least one recombinant microorganism is selected from Escherichia coli, preferably E. coli BL21, E. coli MG1655, E. coli W3110 and their derivatives, Bacillus spp, preferably B. licheniformis, B. subitilis or B. amyloliquefaciens, or derivatives thereof.

25. The method of claim 24, wherein the at least one recombinant microorganism is selected from Saccharomyces spp, Hansenula spp., Komagatella spp, Kluyveromyces spp, or derivatives thereof.

26. The method according to claim 14, wherein at least one NADH oxidase and/or at least one lipase is additionally provided.

27. The method of claim 26, wherein the at least one NADH oxidase comprises an amino acid sequence selected from SEQ ID NO: 5 or SEQ ID NO: 6, or an amino acid sequence having a similarity of 90% or more to SEQ ID NO: 5 or SEQ ID NO: 6.

28. The method according to claim 26, wherein the at least one lipase is a commercial lipase selected from Candida antarctica, Aspergillus niger, Rhizopus oryzae, Penicillium camembertii, Mucor juvanicus, Penicillium roqueforti, porcine pancreas, Candida rugosa, Rhizomucor miehei, Candida antarctica, or Rhizopus delemar.

29. The method according to claim 14, wherein the at least one aldehyde dehydrogenase comprises an amino acid sequence selected from SEQ ID NOs: SEQ ID NO: 9, SEQ ID NO: 10, SEQ ID NO: 11, SEQ ID NO: 12, SEQ ID NO: 13, SEQ ID NO: 14 or SEQ ID NO: 15 or an amino acid sequence having a similarity of 90% or more to SEQ ID NOs: 9, SEQ ID NO: 10, SEQ ID NO: 11, SEQ ID NO: 12, SEQ ID NO: 13, SEQ ID NO: 14 or SEQ ID NO: 15.

30. A vector system comprising at least one vector or plasmid vector comprising:
a nucleic acid segment (a) comprising at least one gene coding for an alpha-dioxygenase having a nucleic acid sequence according to SEQ ID NO: 3 or SEQ ID NO: 4 or a nucleic acid sequence having a similarity of 90% or more to SEQ ID NO: 1 or SEQ ID NO: 2;
a nucleic acid segment (b) comprising at least one gene coding for an aldehyde dehydrogenase; and
optionally, as part of the nucleic acid segment (a) and/or (b), a nucleic acid segment comprising at least one gene coding for an NADH oxidase and/or a nucleic acid segment comprising at least one gene coding for a lipase, wherein the nucleic acid segment (a) and/or the nucleic acid segment (b) is provided on the same vector, or on two or more separate vectors.

31. The vector system according to claim 30, wherein the vector system is a plasmid vector system,
